(19) United States
(12) Patent Application Publication
(10) Pub. No.: US 2024/0207193 A1
(43) Pub. Date: Jun. 27, 2024

(54) CHEMICAL COMPOSITION FOR ENTERIC DRUG DELIVERY

(71) Applicant: BIONXT SOLUTIONS INC., Vancouver (CA)

(72) Inventors: Gordon Lewis AMIDON, Glen Arbor, MI (US); Peter Rolf Werner LANGGUTH, Biebergemund (DE); Jozef AL-GOUSOUS, Mainz (DE); Raimar LOEBENBERG, Edmonton (CA); Daniela Ameral SILVA, Vitória (BR)

(21) Appl. No.: 18/600,459

(22) Filed: Mar. 8, 2024

Related U.S. Application Data
(63) Continuation of application No. PCT/CA2023/050067, filed on Jan. 20, 2023.
(60) Provisional application No. 63/266,962, filed on Jan. 20, 2022.

(51) Int. Cl. A61K 9/28; A61K 45/06

(52) U.S. Cl. CPC A61K 9/2886; A61K 9/2813; A61K 9/2826; A61K 9/2893; A61K 45/06

(57) ABSTRACT

The present disclosure relates to a chemical composition and a method of manufacturing thereof. The chemical composition comprises: (a) a first component; (b) a second component surrounding the first component and enclosing the first component; and (c) an enteric coating forming an exterior of the chemical composition and enclosing the second component and the first component. The second component can consist essentially of a disintegrant; the disintegrant can comprise one or more salts selected from the group consisting of a carbonate salt, a bicarbonate salt, a hydrogendicarbonate salt, a hydrate of any one of the carbonate salt, the bicarbonate salt and the hydrogendicarbonate salt, and any combination thereof. The carbonate salt can be selected from the group consisting of sodium carbonate and potassium carbonate. The bicarbonate salt can be selected from the group consisting of sodium bicarbonate and potassium bicarbonate.

### CROSS-REFERENCE TO RELATED APPLICATIONS

This application is a continuation of Patent Cooperation Treaty (PCT) application No. PCT/CA2023/050067 having an international filing date of Jan. 20, 2023. PCT application No. PCT/CA2023/050067 in turn claims priority from U.S. patent application No. 63/266,962 filed Jan. 20, 2022. All of the applications referred to in this paragraph are hereby incorporated herein by reference.

### TECHNICAL FIELD

The present disclosure relates to a chemical composition for enteric drug delivery and a method of manufacturing thereof.

### BACKGROUND

The formulation of a chemical composition for enteric drug delivery, with attention on avoiding premature dissolution of the chemical composition in the stomach owing to the chemical composition's interaction with stomach acid and timely releasing the active ingredient of the chemical composition in the bicarbonate-based environment of the small intestine, has long been a point of research and development for drug manufacturers.

Many existing chemical formulations are designed to be pH sensitive (see, for example, Bruce et al., International Journal of Pharmaceutics, 2003, 264(1-2): 85-96, Theismann et al., International Journal of Pharmaceutics, 2019, 564: 472-484, PCT/EP2017/058733, PCT/EP2017/058741, and U.S. Pat. No. 9,814,681). For example, some enteric coatings like methacrylic acid based co-polymer coatings specify the pH range between which such enteric coatings will begin to open. However, it has been noted that pH sensitivity may not be the only consideration as to when an enteric coating ruptures or dissolves; that is, other factors in the environment in which the enteric coating is present also affect when an enteric coating ruptures or dissolves. In “Polymers for Controlled Drug Delivery” edited Peter J. Tarcha (see “Polymers for Controlled Drug Delivery” edited by Peter J. Tarcha, Chapter 3 “Polymers for Enteric Coating Application” by George A. Adgyllirah and Gilbert S. Banker, 1991, citing Kanig, Joseph L., Drug Stand., 1954, 22: 113), different enteric coating materials are discussed, including cellulose acetate phthalate (CAP). CAP was noted to be permeable to water vapour, gastric fluid and some ionic substances; as a result, such coating often experienced premature rupturing.

In Ichikawa et al. (1991), “A new multiple-unit oral floating dosage system. I: Preparation and in vitro evaluation of floating and sustained-release characteristics”, Journal of Pharmaceutical Sciences, 80(11): 1062-1066, a chemical composition comprising (i) an effervescent layer containing both sodium bicarbonate and tartaric acid, and (ii) an outer layer comprising a membrane layer consisting essentially of polyvinyl acetate and purified shellac, was described. However, when the composition was immersed in water, the composition swelled which led to untimely rupturing of the outer layer. It is suspected that the untimely rupturing was due at least in part to the evolution of carbon dioxide gas generated by the neutralization reaction between bicarbonate and tartaric acid in the effervescent layer.

In Kim et al., Drug design, Development and Therapy, 2017, 11: 45, a double layer-coated colon-specific drug delivery system comprising a chitosan based polymeric subcoating of the core tablet containing citric acid for microclimate acidification, followed by an enteric coating, was reported. The system showed drug release in a controlled manner by inhibiting drug release in the stomach and intestine, but also releasing the drug gradually in the colon (and not just in the small intestine).

A challenge with the formulation of chemical compositions for enteric drug delivery is avoiding untimely rupturing of said chemical compositions and releasing the active ingredients in said chemical compositions at their desired locations in a subject's gastro-intestinal system.

### SUMMARY

The present disclosure relates to a chemical composition for enteric drug delivery and a method of manufacturing thereof.

According to a part of the disclosure, there is described a chemical composition comprising: (a) a first component; (b) a second component surrounding the first component thereby enclosing the first component therein; and (c) an enteric coating forming an exterior of the chemical composition thereby enclosing the second component therein.

The first component may comprise an active ingredient.

The second component may consist essentially of a disintegrant. The disintegrant may comprise one or more salts selected from the group consisting of a carbonate salt, a bicarbonate salt, a carbonate hydroxide salt, a bicarbonate hydroxide salt, a hydrogendicarbonate salt, a hydrate of any one of the carbonate salt, the bicarbonate salt, a carbonate hydroxide salt, a bicarbonate hydroxide salt, and the hydrogendicarbonate salt, and any combination thereof.

The ratio of the weight of the one or more salts to the weight of the chemical composition may be about 1:100 to about 1:25. The ratio of the weight of the one or more salts to the weight of the chemical composition may be 1:100+. For example, and depending on the size and shape of the chemical composition, the ratio of the weight of the one or more salts to the weight of the chemical composition may be about 1:125, about 1:150, about 1:175, about 1:200, about 1:225 or about 1:250.

The carbonate salt may be selected from the group consisting of alkali metal carbonates, alkaline earth metal carbonates, transition metal carbonates, and any combination thereof. Alkali metal carbonates may be selected from the group consisting of Li₂CO₃, Na₂CO₃, and K₂CO₃. Alkaline earth metal carbonates may be selected from the group consisting of MgCO₃, and CaCO₃. Transition metal carbonates may be selected from the group consisting of FeCO₃, and ZnCO₃.

The bicarbonate salt may be selected from the group consisting of alkali metal bicarbonates, alkaline earth metal bicarbonates, and any combination thereof. Alkali metal bicarbonates may be selected from the group consisting of LiHCO₃, NaHCO₃ and KHCO₃. Alkaline earth metal carbonates may be selected from the group consisting of Mg(HCO₃)₂ and Ca(HCO₃)₂.

The hydrogendicarbonate salt may be selected from an alkali metal hydrogendicarbonate. The alkali metal hydrogendicarbonate may be selected from the group consisting of tri-lithium hydrogendicarbonate, tri-sodium hydrogendicarbonate, and tri-potassium hydrogendicarbonate.

The disintegrant may further comprises one or more polymers. Suitable polymers include hydroxypropyl methylcellulose hydroxyethyl cellulose, methylcellulose, methylhydroxyethylcellulose, ethylcellulose, sodium carboxymethylcellulose, polyvinyl pyrrolidone, polyvinyl alcohol, polyvinyl pyrrolidone-polyvinyl acetate copolymers, polyvinyl alcohol-polyethylene glycol copolymers, polyethylene glycols, methacrylate aminoester copolymer, ethylacrylate-methylmethacrylate copolymer, maltodextrin, and polydextrose.

The weight ratio of the one or more salts to the one or more polymers may be about 1:7 to about 3:10.

According to another part of the disclosure, there is described a method of manufacturing the chemical composition described above, the method comprising: (a) applying the second component onto the first component by spraying; (b) drying the second component; (c) applying the enteric coating onto the second component by spraying; and (d) drying the enteric coating.

The second component may be dried by any one of air-drying and blow-drying. The enteric coating is dried by any one of air-drying and blow-drying.

This summary does not necessarily describe the entire scope of all aspects of the disclosure. Other aspects, features and advantages will be apparent to those of ordinary skill in the art upon review of the following description of specific embodiments.

### BRIEF DESCRIPTION OF THE DRAWINGS

In the accompanying drawings, which illustrate one or more embodiments:

FIG. 1 is a schematic diagram of a chemical composition comprising a first component, a second component, and an enteric coating, according to an embodiment of the chemical composition disclosed herein.

FIG. 2 is a schematic diagram of the chemical composition depicted in FIG. 1 in the presence of an acidic environment.

FIG. 3 is a schematic diagram of the chemical composition depicted in FIG. 1 in the presence of a chemically buffered environment.

FIG. 4 is a schematic diagram of the chemical composition depicted in FIG. 1, wherein the enteric coating is rupturing or has ruptured in a chemically buffered environment.

FIG. 5a is a graph depicting the dissolution of a chemical composition according to another embodiment in an acid environment.

FIG. 5b is a graph depicting the dissolution of the chemical composition of FIG. 5a in two different buffer environments: (i) bicarbonate buffer (5 mM) at pH 6.5; and (ii) phosphate buffer (50 mM) at pH 6.8.

FIG. 6a is a graph depicting the dissolution of a chemical composition according to yet another embodiment in an acid environment.

FIG. 6b is a graph depicting the dissolution of the chemical composition of FIG. 6a in two different buffer environments: (i) bicarbonate buffer (5 mM) at pH 6.5; and (ii) phosphate buffer (50 mM) at pH 6.8.

FIG. 7a is a graph depicting the dissolution of a chemical composition according to yet another embodiment in an acid environment.

FIG. 7b is a graph depicting the dissolution of the chemical composition of FIG. 7a in two different buffer environments: (i) bicarbonate buffer (5 mM) at pH 6.5; and (ii) phosphate buffer (50 mM) at pH 6.8.

FIG. 8a is a graph depicting the dissolution of a chemical composition according to yet another embodiment in an acid environment.

FIG. 8b is a graph depicting the dissolution of the chemical composition of FIG. 8a in two different buffer environments: (i) bicarbonate buffer (5 mM) at pH 6.5; and (ii) phosphate buffer (50 mM) at pH 6.8.

FIG. 9a is a graph depicting the dissolution of a chemical composition according to yet another embodiment in an acid environment.

FIG. 9b is a graph depicting the dissolution of the chemical composition of FIG. 9a in a buffer environment: (i) bicarbonate buffer (5 mM) at pH 6.5.

### DETAILED DESCRIPTION

Directional terms such as “top”, “bottom”, “upwards”, “downwards”, “vertically”, and “laterally” are used in the following description for the purpose of providing relative reference only, and are not intended to suggest any limitations on how any article is to be positioned during use, or to be mounted in an assembly or relative to an environment. Any element expressed in the singular form also encompasses its plural form. Any element expressed in the plural form also encompasses its singular form. The use of the word “a” or “an” when used herein in conjunction with the term “comprising” may mean “one”, but it is also consistent with the meaning of “one or more”, “at least one”, and “one or more than one”.

As used herein, the term “about”, when followed by a recited value, means plus or minus 10% of the recited value.

As used herein, the term “active ingredient” refers to any ingredient that provides biologically active or other direct effect on a subject (e.g., human or other animal).

As used herein, the term “bicarbonate hydroxide salt” means a chemical compound having a formula of Mₓ(HCO₃)_{y}(OH)_{z}, wherein “M” is a metal and wherein each of “x”, “y”, and “z” is an integer that is greater than zero.

As used herein, the term “carbonate hydroxide salt” means a chemical compound having a formula of Mₓ(CO₃)_{y}(OH)_{z}, wherein “M” is a metal and wherein each of “x”, “y”, and “z” is an integer that is greater than zero.

As used herein, the terms “comprising”, “having”, “including”, and “containing”, and grammatical variations thereof, are inclusive or open-ended and do not exclude additional, un-recited elements and/or method steps.

As used herein, the term “consisting essentially of” when used herein in connection with a composition, use or method, denotes that additional elements, method steps or both additional elements and method steps may be present, but that these additions do not materially affect the manner in which the recited composition, method or use functions.

As used herein, the term “consisting of” when used herein in connection with a composition, use or method, excludes the presence of additional elements and/or method steps.

As used herein, the term “disintegrant” means any material that is added to a chemical composition for the purposes of aiding or enhancing the disintegration or rupturing of an enteric coating of the chemical composition.

As used herein, the term “enclosing” means covering all or substantially all of.

As used herein, the term “enteric drug delivery” means the release or delivery of an active ingredient from a chemical composition in the small intestine.

As used herein, the term “HPMC” means hydroxypropyl methyl cellulose.

As used herein, the term “predominant” means “the largest component of”.

One or more embodiments of a chemical composition for enteric drug delivery and one or more embodiments of a method of manufacturing a chemical composition for enteric drug delivery are described in the present disclosure. According to some embodiments, the chemical composition comprises: (a) a first component; (b) a second component surrounding the first component thereby enclosing the first component therein; and (c) an enteric coating forming an exterior of the chemical composition thereby enclosing the second component therein. According to some embodiments, the second component consists essentially of a disintegrant.

The disintegrant comprises one or more salts selected from the group consisting of a carbonate salt, a bicarbonate salt, a carbonate hydroxide salt, a bicarbonate hydroxide salt, a hydrogendicarbonate salt, a hydrate of any one of the carbonate salt, the bicarbonate salt, the carbonate hydroxide salt, the bicarbonate hydroxide salt and the hydrogendicarbonate salt, and any combination thereof. The carbonate salt can be any suitable carbonate salt. Non-limiting examples of suitable carbonate salts include lithium carbonate, sodium carbonate, potassium carbonate, calcium carbonate, magnesium carbonate, iron carbonate and zinc carbonate; sodium carbonate and potassium carbonate are preferred. The bicarbonate salt can be any suitable bicarbonate salt. Non-limiting examples of suitable bicarbonate salts include lithium bicarbonate, sodium bicarbonate, potassium bicarbonate, calcium bicarbonate, and magnesium bicarbonate; sodium bicarbonate and potassium bicarbonate are preferred. The carbonate hydroxide salt can be any suitable carbonate hydroxide salt. The bicarbonate hydroxide salt can be any suitable bicarbonate hydroxide salt. The hydrogendicarbonate salt can be any suitable hydrogendicarbonate salt. Non-limiting examples of suitable hydrogendicarbonate salts include alkali metal hydrogendicarbonate salts. The alkali metal hydrogendicarbonate salt can be selected from the group consisting of tri-lithium hydrogendicarbonate, tri-sodium hydrogendicarbonate, and tri-potassium hydrogendicarbonate; tri-sodium hydrogendicarbonate is preferred.

The disintegrant can further comprise a polymeric material. The one or more salts can be dispersed among the polymeric material. The polymeric material can be a neutral polymeric material. The polymeric material can be a non-neutral polymeric material. The polymeric material can be a cellulosic material. Non-limiting examples of suitable polymeric materials include hydroxypropyl methylcellulose hydroxyethyl cellulose, methylcellulose, methylhydroxyethylcellulose, ethylcellulose, sodium carboxymethylcellulose, polyvinyl pyrrolidone, polyvinyl alcohol, polyvinyl pyrrolidone-polyvinyl acetate copolymers, polyvinyl alcohol-polyethylene glycol copolymers, polyethylene glycols, methacrylate aminoester copolymer, ethylacrylate-methylmethacrylate copolymer, maltodextrin, and polydextrose. Non-limiting examples of suitable cellulosic materials include HPMC, hydroxyethyl cellulose, methylcellulose, methylhydroxyethylcellulose, ethylcellulose, and sodium carboxymethylcellulose. According to some embodiments, the disintegrant does not comprise a polymeric material.

Carbonates and bicarbonates react with aqueous media according to the following equilibrium reactions:

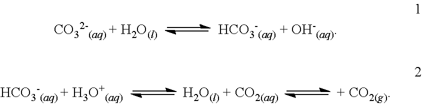

It is believed that the rupturing or the disintegration of the enteric coating of the chemical composition is not only aided or enhanced by (i) the evolution of carbon dioxide gas in the second component while interacting with a subject's gastro-intestinal system, but is also aided or enhanced by (ii) a buffer system that is generated in situ under the enteric coating as the chemical composition interacts with the subject's gastro-intestinal system. Furthermore, it is believed that, as the enteric coating ruptures or disintegrates, water penetrates the second component of the chemical composition causing the chemical composition as a whole to swell, thereby applying additional mechanical force against the enteric coating and causing it to rupture or disintegrate further.

It is also believed that rupturing or disintegration of the enteric coating of the chemical composition at least in part depends on buffer molarity and not just bulk medium pH level. Therefore, it is believed that a molarity threshold (instead of a pH threshold) should be taken into consideration when formulating a chemical composition described herein.

### Chemical Composition

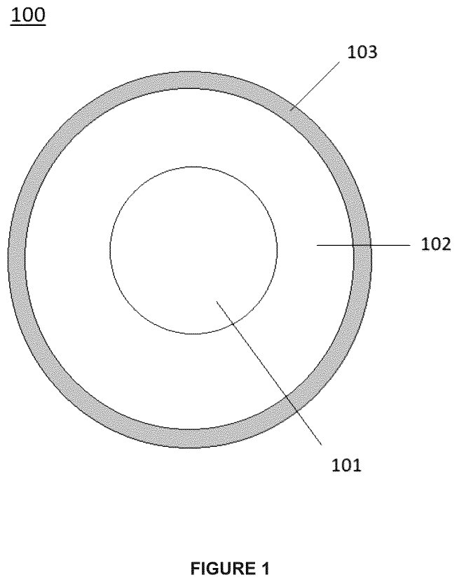

Referring to FIG. 1, and according to an embodiment of the chemical composition disclosed herein, there is a chemical composition 100 comprising: (a) a first component 101; (b) a second component 102 surrounding the first component 101, thereby enclosing the first component 101 therein; and (c) an enteric coating 103 forming an exterior of the chemical composition 100 and surrounding the second component 102 thereby enclosing the second component 102 therein.

First component 101 can comprise an active ingredient or can consist essentially of an active ingredient. First component 101 can be prepared in any suitable form known in the art. Non-limiting examples of suitable forms include capsules, tablets, pellets, granules, and the like.

Second component 102 is coated over first component 101, such that second component 102 surrounds and encloses first component 101. As contemplated in this embodiment, second component 102 consists essentially of a disintegrant. The disintegrant comprises a carbonate salt, a bicarbonate salt, or a combination thereof. The carbonate salt can be any suitable carbonate salt. Non-limiting examples of suitable carbonate salts include sodium carbonate, potassium carbonate, calcium carbonate, and magnesium carbonate; sodium carbonate and potassium carbonate are preferred. The bicarbonate salt can be any suitable bicarbonate salt. Non-limiting examples of suitable bicarbonate salts include sodium bicarbonate, potassium bicarbonate, calcium bicarbonate, and magnesium bicarbonate; sodium bicarbonate and potassium bicarbonate are preferred.

The ratio of the weight of the one or more salts in the disintegrant to the weight of the chemical composition is about 1:100 to about 1:25. For example, the weight ratio of the one or more salts in the disintegrant to the chemical composition can be about 1:100, about 1:95, about 1:90, about 1:85, about 1:80, about 1:75, about 1:70, about 1:65, about 1:60, about 1:55, about 1:50, about 1:45, about 1:40, about 1:35, about 1:30, and about 1:25. In other embodiments, the ratio of the weight of the one or more salts in the disintegrant to the weight of the chemical composition can be different and at least in part depends on the shape and size of the composition. For example, in some embodiments, the ratio of the weight of the one or more salts to the weight of the chemical composition can be 1:100+. Depending on the size and shape of the chemical composition, the ratio of the weight of the one or more salts to the weight of the chemical composition can be about 1:125, about 1:150, about 1:175, about 1:200, about 1:225, or about 1:250.

Enteric coating 103 is a coating that is designed to withstand digestion in the stomach of a subject. Enteric coating 103 can be selected from a suitable coating known in the art. As contemplated in this embodiment, enteric coating 103 is a EUDRAGIT™ coating. In other embodiments, the enteric coating can be another suitable coating. Non-limiting examples of other suitable coatings include cellulose acetate phthalate, cellulose acetate trimellitate, hydroxypropyl methylcellulose acetate succinate, hydroxypropyl methylcellulose phthalate, polyvinyl acetate phthalate, methacrylic acid co-methyl methacrylate, and shellac.

While the embodiment of the chemical composition is depicted in FIG. 1 as having a single first component 101, a single second component 102, and a single enteric coating 103, it will be appreciated by a person having ordinary skill in the art that other embodiments of the chemical composition comprising more than one first component, more than one second component, or more than one enteric coating are possible.

### Manufacturing the Chemical Composition

First component 101 can be prepared into any suitable form known in the art, provided that such suitable form retains a defined shape. Non-limiting examples of suitable forms include capsules, tablets, pellets, granules, and the like.

Second component 102 is applied onto first component 101 by spraying second component 102 onto first component 101 according to a method known in the art. In some embodiments, second component 102 is air-dried. In other embodiments, the second component is blow-dried by hot-air. In other embodiments, the second component is blow-dried by cold-air. In other embodiments, other suitable methods of applying the second component onto the first component may be used. In other embodiments, other suitable methods of drying the second component may be used.

Enteric coating 103 is applied onto second component 102 by spraying enteric coating 103 onto second component 102 according to a method known in the art. In some embodiments, enteric coating 103 is air-dried. In other embodiments, the enteric coating is blow-dried by hot-air. In other embodiments, the enteric coating is blow-dried by cold-air. In other embodiments, other suitable methods of applying the enteric coating onto the second component may be used. In other embodiments, other suitable methods of drying the enteric coating may be used.

### The Chemical Composition: Post-Ingestion

As contemplated herein, the chemical composition is orally ingested by a subject.

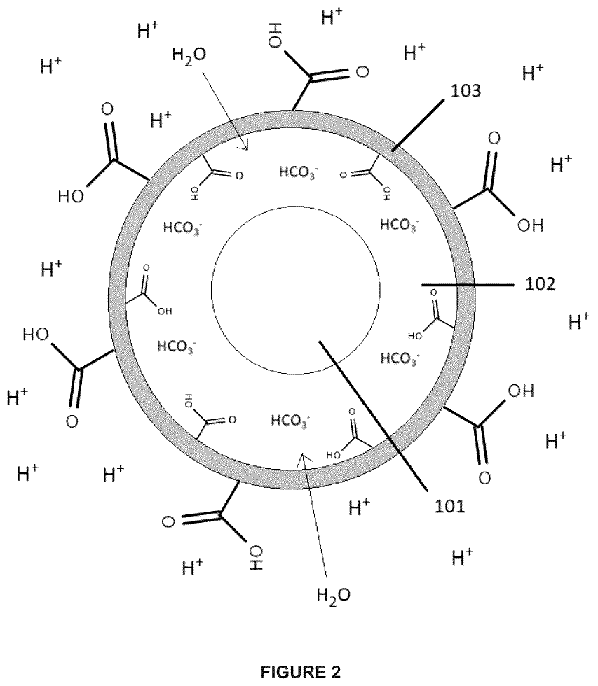

Referring to FIG. 2, chemical composition 100 is depicted in an acidic environment (e.g., the stomach of a subject upon exposure to stomach acids). Enteric coating 103 is not intended to rupture or disintegrate in such an acidic environment, and it is not expected that aqueous media will permeate through enteric coating 103 and into second component 102 and react with the one or more salts in second component 102 to form a buffer layer or carbon dioxide gas.

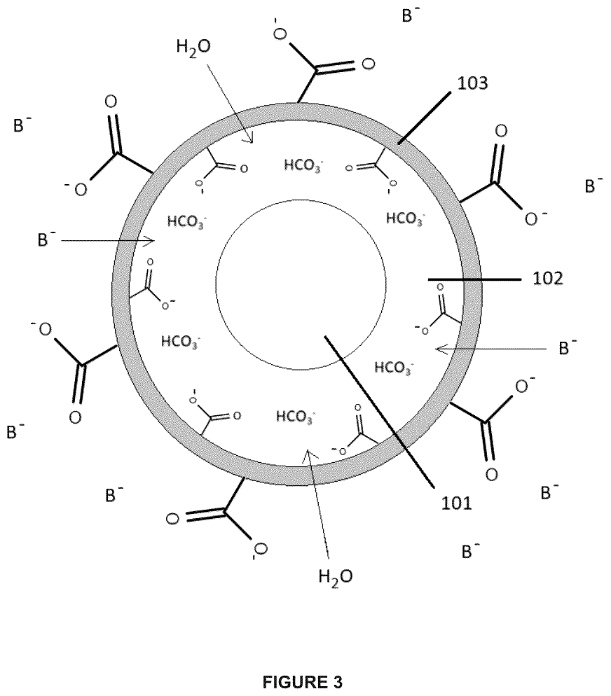

Referring to FIG. 3, chemical composition 100 is depicted in a buffer environment (e.g., the duodenum of a subject). Enteric coating 103 is intended to rupture or disintegrate in such an environment. Referring to FIG. 3, it is believed that the change in pH in the buffer medium (B) of the buffer environment promotes ionization of polymer on the outer side of enteric coating 103; however, the lower buffer capacity of the intestinal bicarbonate buffer at the solid-liquid interface causes the neutralization of enteric coating 103 to occur at a rate that is not conducive to promoting prompt dissolution of enteric coating 103. As such, it is believed that carbonate (CO₃²⁻) or bicarbonate (HCO₃⁻) in second component 102 further causes enteric coating 103 to rupture and disintegrate from the inner side of enteric coating 103. In particular, it is believed that water (H₂O), protons (H⁺) and the buffer species from the buffer environment all permeate through enteric coating 103. Carbonate and bicarbonate in second component 102 reacts with the protons and water that have permeated through enteric coating 103 and into chemical composition 100, and such reaction results in an in situ buffer system that increases the buffer molarity and creates a favorable pH and buffer conditions under enteric coating 103 to promote the dissolution thereof. In addition, CO_{2(g)} is generated as a result of such reaction, and it is believed that the mechanical pressure within chemical composition 100 that is created by such evolution of carbon dioxide gas further aids in rupturing enteric coating 103.

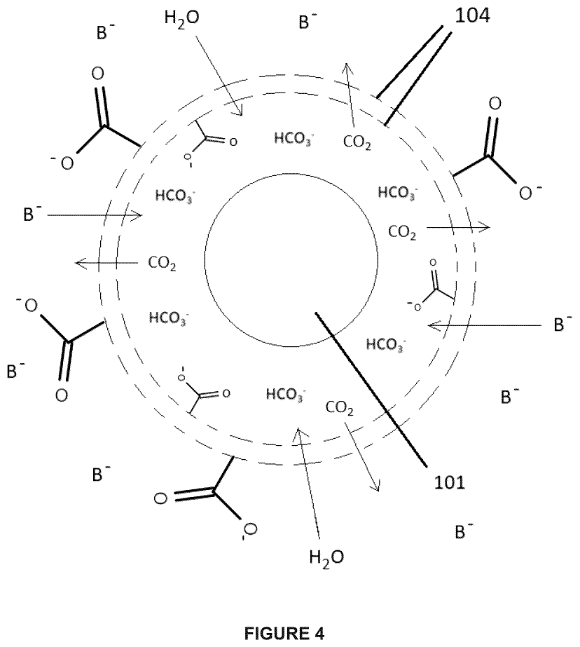

Referring to FIG. 4, enteric coating 103 is depicted as ruptured, resulting in the formation of one or more openings 104 in enteric coating 103. Openings 104 permit additional aqueous media to permeate into chemical composition 100, leading to the eventual disintegration of first component 101 (and to the subsequent release of active ingredient (if present) from first component 101).

### EXAMPLES

Below are several provided non-limiting examples of chemical compositions intended to illustrate specific embodiments of chemical compositions disclosed herein.

Chemical compositions each comprising a first component and an enteric coating enclosing the chemical composition are disclosed. Some chemical compositions disclosed in these examples also comprise a second component enclosing the first component, wherein the enteric coating encloses the second component.

The first component consists essentially of a sugar bead loaded with blue dye, such that when the sugar bead dissolves, the blue dye is released thereby making dissolution of the first component apparent. The chemical compositions were subjected to acid environment tests (e.g., two hours in HCl_{(aq)} 0.1M) to simulate stomach acid environment and to different buffer environments to simulate in vitro environments and the intestinal environment.

The enteric coating comprise: (i) EUDRAGIT™ L 30 D-55 (composition: methacrylic acid and ethyl acrylate copolymer dispersion)—9.3% (w/w) applied (calculated based on dry substance); and (ii) PlasACRYL™ HTP20 (plasticizer)—20% (w/w) applied (calculated based on dry polymer substance).

Some examples of chemical compositions also comprise a second component, such second component comprising HPMC.

The notation “PB” in the Figures refers to “phosphate buffer” and the notation “BCB” in the Figures refers to “bicarbonate buffer”.

### Example 1

A chemical composition comprising a first component and an enteric coating applied thereon is described. No second component is present in this chemical composition.

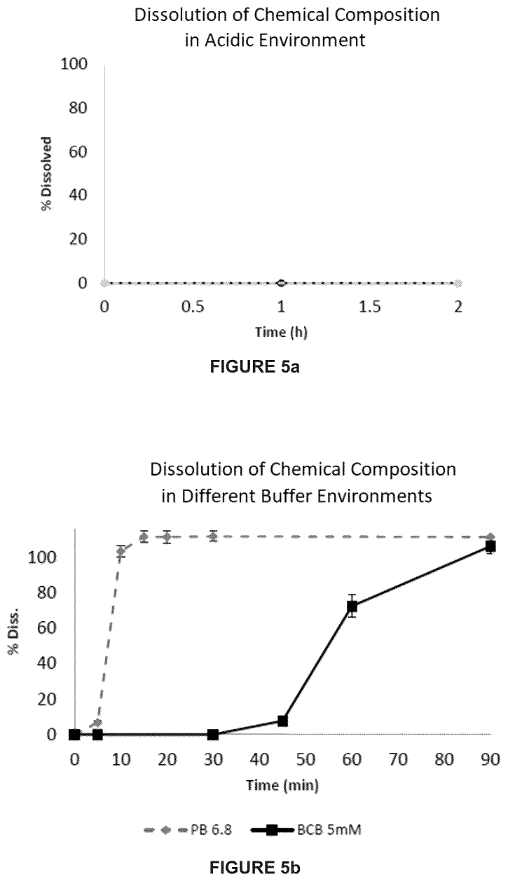

The chemical composition was subjected to an acid environment test. As shown in FIG. 5a, the chemical composition experienced no dissolution in the acid environment test.

The chemical composition was subjected to two buffer environment tests: (i) bicarbonate buffer (5 mM) at pH 6.5; and (ii) phosphate buffer (50 mM) at pH 6.8. As shown in FIG. 5b, the chemical composition dissolved more readily in a phosphate buffer solution than in a bicarbonate buffer solution, notwithstanding the similarity in pH levels of the two buffer solutions.

### Example 2

A chemical composition comprising a first component, a second component enclosing the first component, and an enteric coating enclosing the second component is described. The second component comprised bicarbonate (1.3%) dissolved in HPMC (7.75%) (w/w). The second component was sprayed onto the first component and air-dried at room temperature overnight. The enteric coating was then sprayed onto the dried second component, and the enteric coating was air-dried at 40° C. overnight.

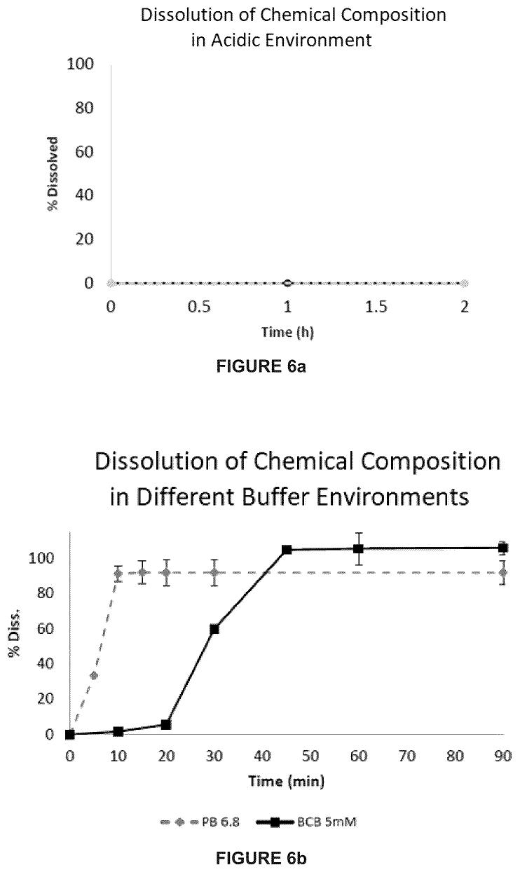

The chemical composition was subjected to an acid environment test. As shown in FIG. 6a, the chemical composition experienced no dissolution in the acid environment test.

The chemical composition was subjected to two buffer environment tests: (i) bicarbonate buffer (5 mM) at pH 6.5; and (ii) phosphate buffer (50 mM) at pH 6.8. As shown in FIG. 6b, the chemical composition dissolved much more readily in a phosphate buffer solution than in a bicarbonate buffer solution, notwithstanding the similarity in pH levels of the two buffer solutions.

### Example 3

A chemical composition comprising a first component, a second component enclosing the first component, and an enteric coating enclosing the second component is described. The second component comprised bicarbonate (2.1%) dissolved in HPMC (7.75%) (w/w). The second component was sprayed onto the first component and air-dried at room temperature overnight. The enteric coating was then sprayed onto the dried second component, and the enteric coating was air-dried at 40° C. overnight.

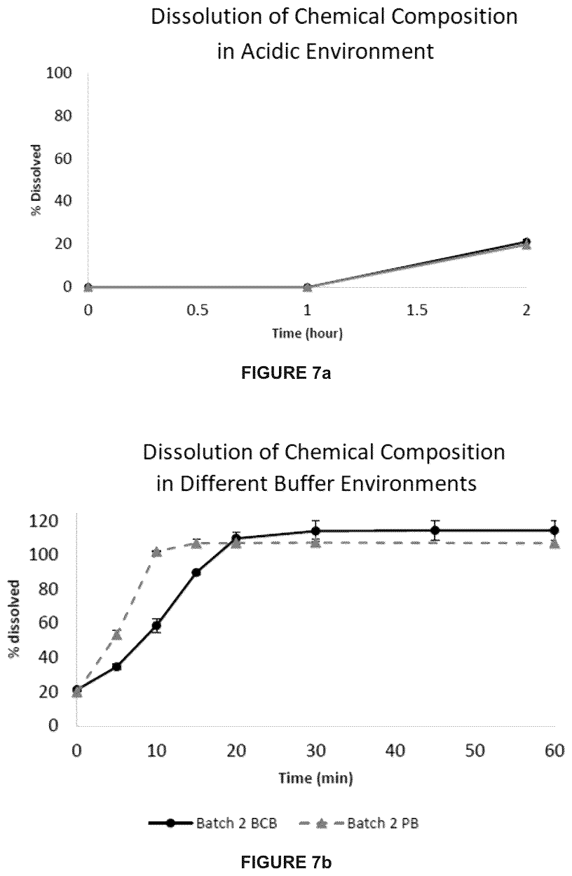

The chemical composition was subjected to an acid environment test. As shown in FIG. 7a, the chemical composition experienced no dissolution in the acid environment test during the first hour of such test. However, the chemical composition exhibited signs of dissolution soon thereafter, and after about two hours of the acid environment test, 20% dissolution (as determined by the amount of blue dye released) was detected.

The chemical composition was subjected to two buffer environment tests: (i) bicarbonate buffer (5 mM) at pH 6.5; and (ii) phosphate buffer (50 mM) at pH 6.8. As shown in FIG. 7b, the chemical composition dissolved quicker in a phosphate buffer solution than in a bicarbonate buffer solution, notwithstanding the similarity in pH levels of the two buffer solutions.

### Example 4

A chemical composition comprising a first component, a second component enclosing the first component, and an enteric coating enclosing the second component is described. The second component comprised phosphate dissolved in HPMC (7.75%) (w/w). The second component was sprayed onto the first component and air-dried at room temperature overnight. The enteric coating was then sprayed onto the dried second component, and the enteric coating was air-dried at 40° C. overnight.

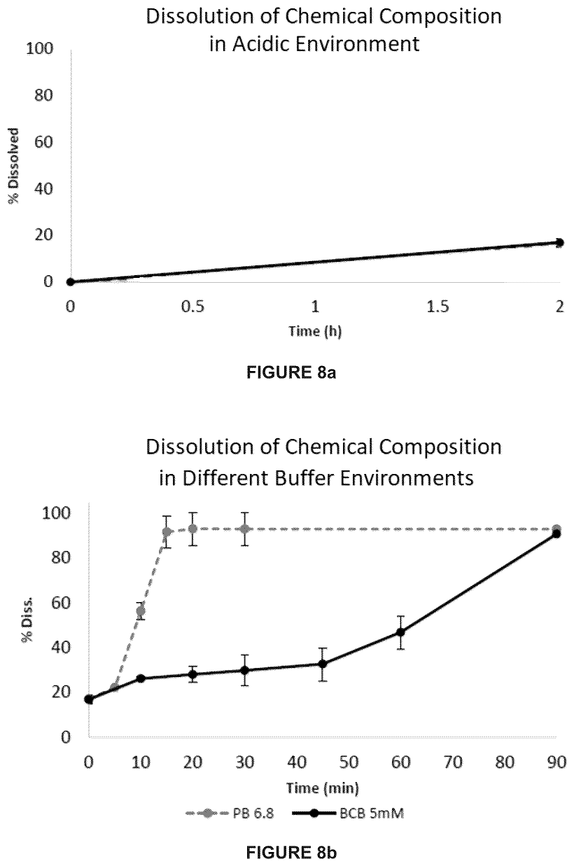

The chemical composition was subjected to an acid environment test. As shown in FIG. 8a, the chemical composition experienced about 5% dissolution (as determined by the amount of blue dye released) within the first hour of the acid environment test. After about two hours of the acid environment test, 17% dissolution (as determined by the amount of blue dye released) was detected.

The chemical composition was subjected to two buffer environment tests: (i) bicarbonate buffer (5 mM) at pH 6.5; and (ii) phosphate buffer (50 mM) at pH 6.8. As shown in FIG. 8b, the chemical composition dissolved quicker in a phosphate buffer solution than in a bicarbonate buffer solution, notwithstanding the similarity in pH levels of the two buffer solutions.

### Example 5

A chemical composition comprising a first component, a second component enclosing the first component, and an enteric coating enclosing the second component is described. The composition of the second component was HPMC. The second component was sprayed onto the first component and air-dried at room temperature overnight. The enteric coating was then sprayed onto the dried second component, and the enteric coating was air-dried at 40° C. overnight.

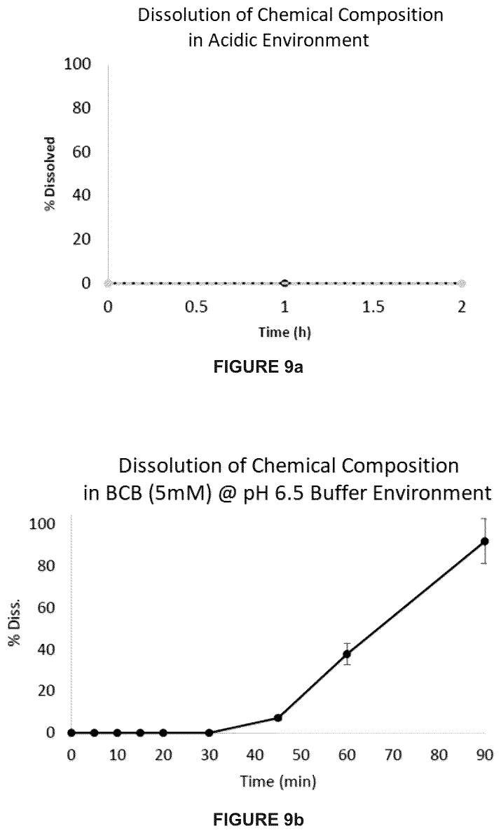

The chemical composition was subjected to an acid environment test. As shown in FIG. 9a, the chemical composition experienced no dissolution in the acid environment test.

The chemical composition was subjected to a buffer environment test: (i) bicarbonate buffer (5 mM) at pH 6.5. As shown in FIG. 9b, the chemical composition dissolved overtime.

Based on the examples disclosed in this present disclosure, it appears that a second component comprising bicarbonate of about 1% to about 2% (w/w), or weight ratio of bicarbonate to the chemical composition is about 1:100 to about 1:50, mitigates at least in part the issues related premature rupturing or disintegration of the enteric coating of chemical compositions intended for enteric drug delivery. In addition, it is believed that the second component comprising bicarbonate assists in opening the enteric coating by creating a region with higher buffer concentration. Moreover, since bicarbonate can react with acid and produces carbon dioxide gas, the release of carbon dioxide gas from the second component further assists in rupturing the enteric coating. As openings in the enteric coating are created, aqueous media penetrates various components of the chemical composition causing them to swell and disintegrate.

### GENERAL

It is contemplated that any part of any aspect or embodiment discussed in this specification can be implemented or combined with any part of any other aspect or embodiment discussed in this specification. While particular embodiments have been described in the foregoing, it is to be understood that other embodiments are possible and are intended to be included herein. It will be clear to any person skilled in the art that modification of and adjustment to the foregoing embodiments, not shown, is possible.

What is claimed is:

1. A chemical composition comprising:
(a) a first component;
(b) a second component surrounding the first component and enclosing the first component, the second component consisting essentially of a disintegrant, the disintegrant comprising one or more salts selected from the group consisting of a carbonate salt, a bicarbonate salt, a hydrogendicarbonate salt, a carbonate hydroxide salt, a bicarbonate hydroxide salt, a hydrate of any one of the carbonate salt, the bicarbonate salt, a carbonate hydroxide salt, a bicarbonate hydroxide salt and the hydrogendicarbonate salt, and any combination thereof; and
(c) an enteric coating forming an exterior of the chemical composition and surrounding and enclosing the second component.

2. The chemical composition as claimed in claim 1, wherein the first component comprises an active ingredient.

3. The chemical composition as claimed in claim 1, wherein the one or more salts is selected from the group consisting of the carbonate salt, the bicarbonate salt, and a combination thereof.

4. The chemical composition as claimed in claim 3, wherein the one or more salts is selected from the group consisting of Na₂CO₃, K₂CO₃, NaHCO₃, KHCO₃, and any combination thereof.

5. The chemical composition as claimed in claim 4, wherein the one or more salts is selected from the group consisting of Na₂CO₃, K₂CO₃, and any combination thereof.

6. The chemical composition as claimed in claim 4, wherein the one or more salts is selected from the group consisting of NaHCO₃ and KHCO₃, and any combination thereof.

7. The chemical composition as claimed in claim 1, wherein the disintegrant further comprises one or more polymers.

8. The chemical composition as claimed in claim 7, wherein at least one of the one or more polymers is a cellulose.

9. The chemical composition as claimed in claim 8, wherein the cellulose is selected from the group consisting of hydroxypropyl methylcellulose, hydroxyethyl cellulose, methylcellulose, methylhydroxyethylcellulose, ethylcellulose, and sodium carboxymethylcellulose.

10. The chemical composition as claimed in claim 9, wherein the cellulose is hydroxypropyl methylcellulose.

11. The chemical composition as claimed in claim 7, wherein a ratio of the weight of the one or more salts to the weight of the one or more polymers is about 1:7 to about 3:10.

12. The chemical composition as claimed in claim 1, wherein a ratio of the weight of the one or more salts to the weight of the chemical composition is about 1:250 to about 1:25.

13. A method of manufacturing the chemical composition as claimed in claim 1, the method comprising:
(a) applying the second component onto the first component by spraying;
(b) drying the second component;
(c) applying the enteric coating onto the second component by spraying; and
(d) drying the enteric coating.

14. The method as claimed in claim 13, wherein the second component is dried by any one of air-drying and blow-drying.

15. The method as claimed in claim 13, wherein the enteric coating is dried by any one of air-drying and blow-drying.
